(19) United States
(12) Patent Application Publication
(10) Pub. No.: US 2021/0052327 A1
(43) Pub. Date: Feb. 25, 2021

(54) BONE REGISTRATION IN TWO-STAGE ORTHOPEDIC REVISION PROCEDURES

(71) Applicant: Think Surgical, Inc., Fremont, CA (US)

(72) Inventors: Kyle Kuznik, Fremont, CA (US); Micah Forstein, Fremont, CA (US); Steve Whiseant, Fremont, CA (US)

(73) Assignee: Think Surgical, Inc., Fremont, CA (US)

(21) Appl. No.: 16/962,012

(22) PCT Filed: Feb. 12, 2019
(86) PCT No.: PCT/US2019/017570

§ 371 Date: Jul. 14, 2020

Related U.S. Application Data
(60) Provisional application No. 62/629,954, filed on Feb. 13, 2018.

(51) Int. Cl. A61B 34/10; A61B 34/37; A61B 34/00; A61B 90/00

(52) U.S. Cl. CPC A61B 34/10; A61B 34/37; A61B 34/76; A61B 90/39; A61B 2090/065; A61B 2090/3916; A61B 2090/3937; A61B 2090/3983; A61B 90/06

(57) ABSTRACT

A method for registering a bone is provided that includes the installation of two or more registration markers on an exposed portion of the bone as part of a first stage of a revision. An image data set of the bone is generated to create imaged registration markers. A surgical plan is generated for implanting one or more secondary implants in a second stage of the two-stage procedure based on the image data. The bone is registered to the surgical plan and a computer-assist device using the two or more registration markers by: digitizing a set of positions of the two or more registration markers to create digitized registration markers; and mapping the set of positions of the digitized registration markers with a corresponding set of positions of the imaged registration markers. A system for performing the method is provided. Implant revision is promoted through the use of the method.

### RELATED APPLICATIONS

This application claims priority benefit of U.S. Provisional Application Ser. No. 62/629,954 filed 13 Feb. 2018; the contents of which are hereby incorporated by reference.

### TECHNICAL FIELD

The present invention generally relates to the field of computer-assisted orthopedic revision surgery, and more particularly to a system and method for registering a bone in two-stage orthopedic revision procedures.

### BACKGROUND

Computer-assisted orthopedic surgery is an expanding field having applications in total joint arthroplasty (TJA), bone fracture repair, maxillofacial reconstruction, and spinal reconstruction. Computer-assisted surgical systems generally aid in the planning and execution of an orthopedic procedure. For example, the TSOLUTION ONE® Surgical System (THINK Surgical, Inc., Fremont, Calif.) aids in the planning and execution of total hip arthroplasty (THA) and total knee arthroplasty (TKA). The TSOLUTION ONE® Surgical System includes: a pre-operative planning software program to generate a surgical plan using an image data set of the patient's bone and computer-aided design (CAD) files of implants; and an autonomous surgical robot that precisely mills the bone to receive an implant according to the surgical plan. In order for the computer-assisted surgical system to accurately prepare a bone, the bone needs to be registered to the surgical system in the operating room. The registration procedure maps the surgical plan onto the position and orientation (POSE) of the bone in the coordinate system the surgical system.

Several registration procedures are known in the art, illustratively including pin-based, point-to-point, point-to-surface, laser scanning, and image registration, as described in U.S. Pat. Nos. 5,951,475, 6,033,415, 8,287,522, and 8,010,177. However, the tasks required to perform these registration procedures are tedious. The most commonly used registration procedure relies on the manual collection of several points on the bone with a tracked digitizer probe. The surgeon is guided to collect specific points on the bone by way of a display monitor. If the surgeon incorrectly collects one or more points and the registration fails, then the entire registration procedure is repeated until the registration passes, thereby increasing the duration of the surgical procedure. Image registration is a viable alternative that uses X-ray imagery or fluoroscopy to register the surgical plan to the bone, but at the expense of exposing the patient to additional radiation. Pin-based registration procedures have likewise been used in the past, which requires the implantation of two or more pins in the patient's bone prior to acquiring the image data set for planning. The pins remain in the bone until the procedure occurs, at which time the pins are exposed and digitized to register the bone. Even though pin-based registration is quick and reliable, the additional pre-operative procedure of pin implantation can be invasive and cause residual pain.

A particular application for registering a bone to a surgical plan and/or computer-assisted surgical system is revision orthopedic procedures. Revision orthopedic procedures are necessary when a primary implant has failed and needs to be replaced. One cause of implant failure is infection. For revision cases due to infection, the surgical team usually performs a two-stage revision procedure. The first stage involves the removal of the primary implant from the bone, after which the patient incision is sewn up and a strict antibiotic regimen is undertaken spanning several weeks until the infection subsides. The patient is typically kept in the hospital during the antibiotic regimen for monitoring. Once the infection has subsided, the second stage commences where the surgical team re-exposes the joint and installs the secondary implants in the bones. In the second stage of a computer-assisted revision procedure, the bones in the joint need to be registered to the revision surgical plan and/or the surgical system. However, much of the bone has been amputated from the primary procedure, reducing the number of viable locations to collect registration points, and ultimately making the registration procedure in revision cases difficult to execute.

Thus, there exists a need in the art for a method and system to efficiently and effectively register a bone for two-stage computer-assisted revision procedures.

### SUMMARY OF THE INVENTION

A method for registering a bone is provided that is advantageous in monitoring healing processes in the bone as well as being an adjunct to implant revision, especially in occurrences of infection associated with the implant. The method includes the installation of two or more registration markers on an exposed portion of the bone as part of a first stage of a revision. An image data set of the bone is generated to create imaged registration markers. A surgical plan is generated for implanting one or more secondary implants in a second stage of the two-stage procedure based on the image data. The bone is registered to the surgical plan and a computer-assist device using the two or more registration markers by: digitizing a set of positions of the two or more registration markers to create digitized registration markers; and mapping the set of positions of the digitized registration markers with a corresponding set of positions of the imaged registration markers.

A method for a two-stage revision procedure as to a failed primary implant in the bone of a patient is provided. A bone is exposed through a surgical incision to gain access to the failed primary implant in a first-stage of the two-stage revision procedure. Thereafter, the primary implant is removed from the bone and two or more registration markers are installed on an exposed portion of the bone before closing the surgical incision to end the first stage of the two-stage revision procedure. An image data set of the bone is generated to create imaged registration markers. A surgical plan is generated for implanting one or more secondary implants in a second stage of the two-stage procedure based on the image data. The bone is then re-exposed to begin a second stage of the two-stage revision procedure and registered to the surgical plan and a computer-assist device using the two or more registration markers by: digitizing a set of positions of the two or more registration markers to create digitized registration markers; and mapping the set of positions of the digitized registration markers with a corresponding set of positions of the imaged registration markers. The bone of the patient is then prepared according to the surgical plan with the aid of the computer-assist device to implant the one or more secondary implants in the bone of the two-stage revision procedure.

A system for performing such methods is also disclosed.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Dimensions of components and features shown in the figures are generally chosen for convenience and clarity of presentation and are not necessarily shown to scale. The figures are listed below.

FIG. 1 depicts a method for registering a bone in two-stage revision procedures in accordance with embodiments of the invention;

FIGS. 2A-2C depict several different registration markers in accordance with embodiments of the invention, where FIG. 2A is a point registration marker, FIG. 2B is a groove recovery marker, and FIG. 2C is a two-point recovery marker;

FIG. 3 depicts a model of a bone being registered to an actual bone in accordance with embodiments of the invention; and

FIG. 4 depicts a computer-assist device for performing a revision joint arthroplasty procedure in accordance with embodiments of the invention.

### DETAILED DESCRIPTION

The present invention has utility as a system and method for registering a bone in two-stage revision procedures. The system and method are particularly advantageous for two-stage revision procedures that require the exposure of the bone at two separate time points, such as two-stage revision procedures caused by infection in a human or mammalian patient. Further, it should be appreciated that although the systems and methods described herein make reference to a revision procedure for a knee replacement, the systems and methods are equally applicable to other bones and joints in the body, illustratively including the hip, ankle, shoulder, maxillofacial, spine, and skull. Additionally, although a joint replacement revision procedure is described herein, other computer-assisted procedures requiring the exposure of a bone at two separate time points is likewise applicable, such as bone fracture repair, maxillofacial reconstruction, and spinal procedures. While the method of the present invention is expected to have the most impact on the treatment of human patients, it is appreciated that the system and method are readily adapted to treat other mammalian patients that illustratively include horses, especially race horses; bovines; felines; canines; and ungulates.

The present invention will now be described with reference to the following embodiments. As is apparent by these descriptions, this invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment may be deleted from the embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references mentioned herein are incorporated by reference in their entirety.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also, as used herein, “and/or” refers to and encompasses any and all possible combinations of one or more of the associated listed items, as well as the lack of combinations when interpreted in the alternative (“or”).

As used herein, the term “registration” refers to the determination of the spatial relationship between two or more objects or coordinate systems such as a computer-assist device and at least one of a bone, or an image data set of a bone. “Re-registration” refers to any subsequent registration procedure that occurs after an initial registration and is executed with the use of the recovery markers.

As used herein, the term “digitizer” refers to a measuring device capable of measuring physical coordinates in three-dimensional space. Examples of a “digitizer” include a high-resolution electro-mechanical sensor arm as described in U.S. Pat. No. 6,033,415, an optically tracked probe as described in U.S. Pat. No. 7,043,961, an end-effector of a robot, and similar measuring devices that may be tracked by other tracking systems known in the art.

As used herein, the term “digitizing” refers to the collection, recordation, or measurement of one or more physical coordinates in three-dimensional space.

As used herein, the term “real-time” refers to a processor in which input data is processed within milliseconds such that calculated values are available within 10 seconds of computational initiation.

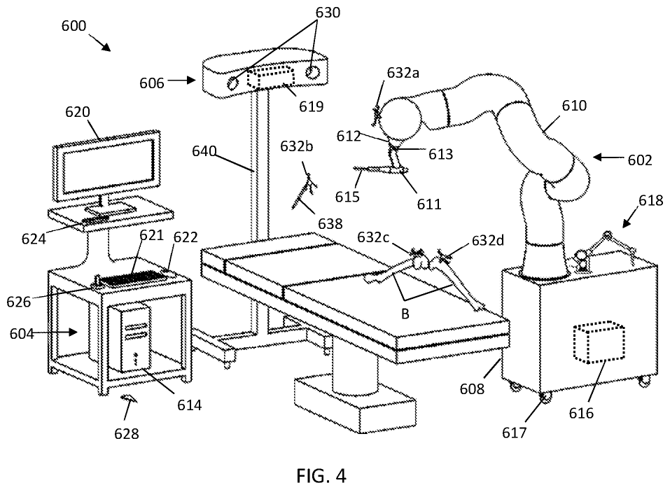

Also, referenced herein are computer-assist devices. Examples of computer-assisted devices illustratively include a passive tool tracked by a tracking system, a one to six plus degree of freedom active hand-held surgical system, a serial-chain robotic system, a parallel robotic system, a master-slave robotic system, or an articulating robotic system assembled directly to a bone, as described in U.S. Pat. Nos. 5,086,401, 6,033,415, 7,206,626, 8,876,830 and 8,961,536, and U.S. Pat. App. Nos. 2013/0060278, and 2005/0216032. The computer-assist devices may provide autonomous, semi-autonomous, haptic, or no (passive) control, or any combination thereof. A specific computer-assist device 600 for performing revision joint arthroplasty procedures is described below with reference to FIG. 4.

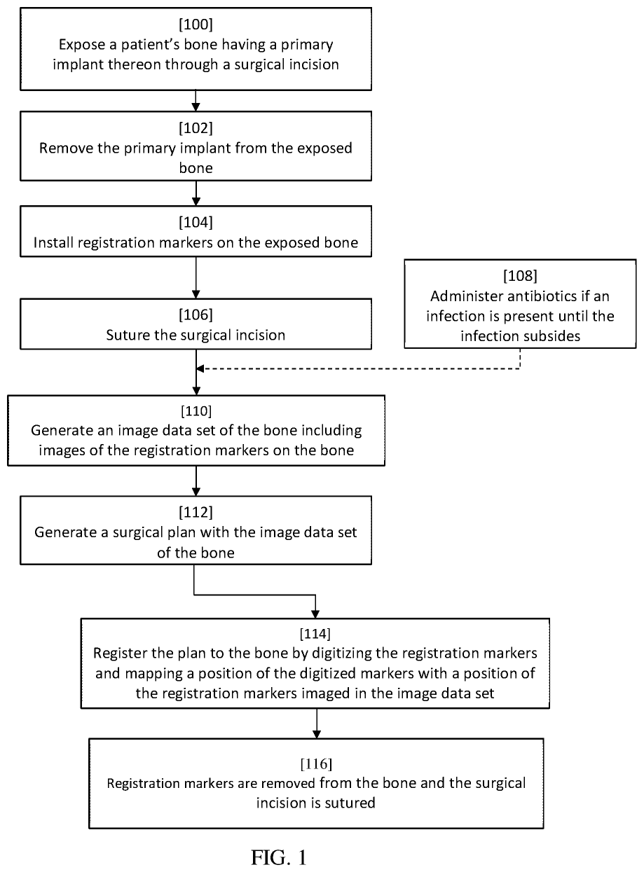

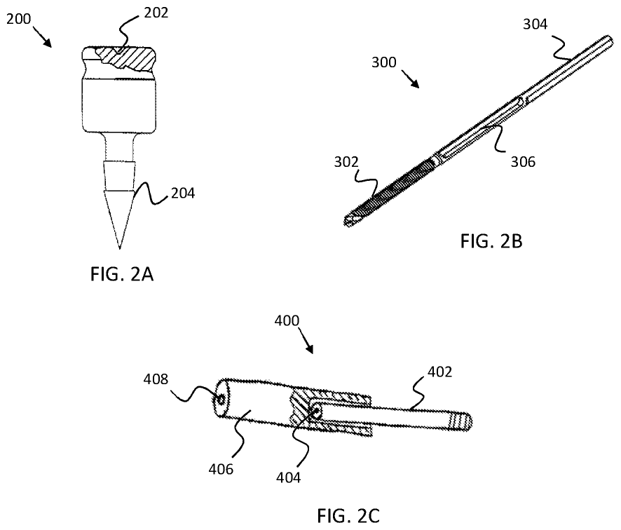

Referring now to the figures, FIG. 1 depicts a particular embodiment of a method for registering a bone in two-stage revision procedures. The first stage begins by exposing the patient's bone through a surgical incision to gain access to the failed primary implant, as shown at Block 100, and removing the primary implant using techniques known in the art, as shown at Block 102. Two or more registration markers (200, 300, 400 as shown in FIGS. 2A to 2C) are then installed on the exposed bone for registering the bone during the second stage of the two-stage revision procedure, as shown at Block 104. The surgical incision is subsequently closed by conventional techniques such as sutures, adhesives, or staples; ending the first stage of the two-stage revision procedure, as shown at Block 106. In the event the primary implant failed due to infection, the patient is administered antibiotics until the infection subsides, as shown at Block 108, which may take several weeks depending on the severity of the infection. It is appreciated that antibiotics, bone growth factors, or other medications are each independently readily administered pre-operatively, between first and second surgical stages, or thereafter. After the first stage and prior to the second stage of the revision procedure, an image data set of the bone is generated, as shown at Block 110 to generate a surgical plan for implanting the secondary implant(s) in the second stage of the two-stage procedure, as shown at Block 112. The image data set includes images of the registration markers to register the bone during the second stage of the revision procedure. The second stage of the two-stage revision procedure begins by re-exposing the affected bone and the marker therein. The bone is then registered to the surgical plan and a computer-assist device using the two or more registration markers (200, 300, 400) by digitizing a position of the registration markers (200, 300, 400) with a digitizer, as described below, and mapping the position of the digitized registration markers with a position of the registration markers imaged in the image data set, as shown at Block 114. It is appreciated that in some inventive embodiments, as shown at 200, and 400; the registration markers have a symmetry axis, while in other embodiments, registration markers that are asymmetric or have only a finite number of planes of symmetry are affixed to the bone of interest. The bone is then prepared according to the surgical plan with the aid of the computer-assist device to implant the secondary implants in the bone. Once the secondary implants are installed, the registration markers (200, 300, 400) are removed from the bone and the surgical incision is sutured, as shown at Block 116. Further details of the embodiments of the method are described below.

With reference to FIGS. 2A to 2C, particular embodiments of registration markers (200, 300, 400) used for registering the bone in two-stage revision cases are shown, where FIG. 2A is a point registration marker 200, FIG. 2B is a groove registration marker, and FIG. 2C is a two-point registration marker 400. The point registration marker 200 includes a divot 202 and a bone-engaging portion 204. The divot 202 facilitates the collection of a single point on the point registration marker 200 with a digitizer. The bone-engaging portion 204 may be a spike, pin, or threaded shaft for inserting the point registration marker 200 in the bone. The groove registration marker 300 is an elongated shaft having a bone-engaging portion 302, a proximal portion 304, and a groove 306 situated between the bone-engaging region 302 and the proximal portion 304. The bone-engaging region 302 may likewise be a spike, pin, or a threaded portion of the shaft for inserting the groove registration marker 300 in the bone. The proximal portion 304 aids a user in inserting the groove registration marker 300 in the bone and may be configured to receive a chuck, collet, or socket of a drill or screwdriver. The groove 306 facilitates the collection of a plurality of points along the length of the groove 306 with a digitizer, where a vector can be fitted to the plurality of points to aid in the registration. The two-point registration marker 400 includes a first portion 402 having a first divot 404 and a second portion 406 having a second divot 408. The first portion 402 may be a shaft having bone-engaging portion at one end for inserting the two-point registration marker 400 in the bone and the first divot 404 positioned along the length of the shaft or at an opposing end of the bone-engaging portion. The second portion 406 may be a cap that removably secures to the first portion 402, by for example, press fitting or threading onto an end of the first portion 402. The first divot 404 and second divot 408 facilitate the collection of two single points on the two-point registration marker 400 with a digitizer. In other embodiments, the two-point registration marker 400 is a single shaft having a bone-engaging portion, a first divot 404 positioned at a first position along the length of the shaft, and a second divot 408 positioned at an end or second position along the length of the shaft.

In order to resolve 6-degrees-of-freedom of the bone to register the bone, at least one of the following combinations of registration markers is preferably installed on the bone: three point registration markers 200; a single point registration marker 200 and a groove recover marker 300; or a point registration marker 200 and a two-point registration marker 400. In other embodiments, two point registration markers 200 may be used if both the position and orientation of the point registration markers 200 can be determined with the digitizer.

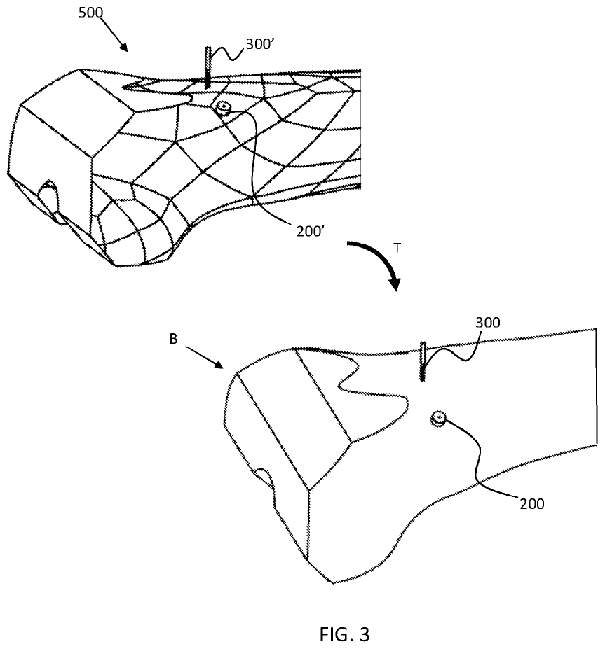

With the registration markers (200, 300, 400) installed, an image data set of the bone and the markers (200, 300, 400) is acquired. The image data set may be acquired with an imaging modality illustratively including computed tomography scan (CT), magnetic resonance imaging (MRI), ultrasound, or X-rays. In a specific inventive embodiment, the image data set is acquired by collecting a plurality of points on the bone with a digitizer to create a point cloud/surface map of the bone during the first stage of the two-stage revision procedure (the installed registration markers (200, 300, 400) likewise being digitized to register the bone in the second stage of the procedure). With reference to FIG. 3, a three-dimensional (3D) model of the bone and the imaged markers 500 may be generated to aid a surgeon in generating a surgical plan. The surgical plan includes the desired position and orientation (POSE) for the secondary implants as planned by a surgeon in a pre-operative planning software program having the 3D model of the bone 500 and computer-aided design (CAD) files of the implants stored therein. However, it should be appreciated that the surgeon may plan the POSE of the secondary implants on any image data set without the need to generate a 3D model of the bone 500.

In the second stage of the two-stage revision procedure, the bone is re-exposed and the registration markers (200, 300, 400) are digitized. The surgical plan, which is tied to the image data set, may then be registered by determining the transformation T between the image data set and the bone B via the markers (200, 300, 400). The transformation T is determined by mapping the position of the digitized registration markers (200, 300, 400) present on the bone with the imaged registration markers (200′, 300′, 400′) in the image data set. More specifically, the points/vectors collected by the digitizer on the registration markers (200, 300, 400) is mapped to the same points/vectors of the imaged registration markers (200′, 300′, 400′). During post-registration, the computer-assist device may aid the surgeon in preparing the bone to receive the secondary implants in the desired POSE according to the surgical plan. In a specific embodiment, the registration markers (200, 300, 400) may act as registration recovery markers during bone preparation to re-register the bone in the event of bone motion. After the bone is prepared, the secondary implants are implanted in/on the bone, the registration markers (200, 300, 400) are removed, and the surgical incision is sutured.

In a specific inventive embodiment, the registration markers (200, 300, 400) further include an antibiotic property, a bone growth property, or a combination thereof. The property may be a material known in the art in which the recovery markers (200, 300, 400) are made of. The property may be created through a coating known in the art that is present on the surface of the registration markers (200, 300, 400). In other embodiments, the registration markers (200, 300, 400) may be porous, having a matrix infused with a therapeutic or prophylactic substance that diffuses therefrom and into the surrounding bone. The antibiotic property is particularly advantageous for treating a patient having a present infection, which may have caused the primary implant to fail.

In a particular inventive embodiment, the registration markers (200, 300, 400) may further be made of a biocompatible material that degrades overtime. In some instances, the registration markers (200, 300, 400) may be installed on the bone at a location that makes it difficult to remove one or more of the registration markers (200, 300, 400) after the secondary implants are installed. The bone may still be registered by digitizing one or more of the registration markers (200, 300, 400) percutaneously, but difficult to extract from the bone. Therefore, the registration markers may naturally dissolve/degrade without having to create a larger incision than necessary and reduce any harm to the patient. In an alternative embodiment, the registration markers (200, 300, 400) are bio-inert and remain in the bone like an implant. In other words, rather than removing the registration markers (200, 300, 400) at the close of the second stage of the procedure, the markers (200, 300, 400) are left in the bone. This is likewise advantageous if the markers (200, 300, 400) were inserted percutaneously in an un-exposed region of the bone.

### Computer-Assist Device

In a particular embodiment, with reference to FIG. 4, the computer-assist device described herein is a robotic surgical system 600. The surgical system 600 generally includes a surgical robot 602, a computing system 604, and includes at least one of a mechanical digitizer 618 or a non-mechanical tracking system 606 (e.g., optical tracking system).

The surgical robot 602 may include a movable base 608, a manipulator arm 610 connected to the movable base 608, an end-effector flange 612 located at a distal end of the manipulator arm 610, and an end-effector assembly 611 for holding and/or operating an end-effector tool 615 removably attached to the flange 612 by way of an end-effector mount 613. A force sensor (not shown) may further be present on or proximal to the end-effector assembly 611 to measure forces experienced on the end-effector tool 615. The movable base 608 in some inventive embodiments includes a set of wheels 617 to maneuver the movable base 608, which may be fixed into position using a braking mechanism such as a hydraulic brake. The manipulator arm 610 includes various joints and links to manipulate the tool 615 in various degrees of freedom. If the mechanical digitizer 618 is not present, the end effector assembly 611 may be fitted with a probe to act as a digitizer directly. The joints are illustratively prismatic, revolute, spherical, or a combination thereof.

The computing system 604 generally includes a planning computer 614; a device computer 616; an optional tracking system computer 619 if a tracking system 606 is present; and peripheral devices. The planning computer 614, device computer 616, and tracking computer 619, may be separate entities, a whole single unit, or combinations thereof depending on the surgical system. The peripheral devices allow a user to interface with the surgical system components and may include: one or more user-interfaces, such as a display or monitor 620; and user-input mechanisms, such as a keyboard 621, mouse 622, pendent 624, joystick 626, foot pedal 628, or the monitor 620 in some inventive embodiments has touchscreen capabilities.

The planning computer 614 contains hardware (e.g., processors, controllers, and memory), software, data and utilities that are in some inventive embodiments dedicated to the planning of a surgical procedure, either pre-operatively or intra-operatively for performing at least one, if not all the steps 110, 112, and 114 with respect to FIG. 1. This may include reading medical imaging data, segmenting imaging data, constructing three-dimensional (3D) virtual models, storing computer-aided design (CAD) files, providing various functions or widgets to aid a user in planning the surgical procedure, and generating surgical plan data. The final surgical plan includes operational data for modifying a volume of tissue that is defined relative to the anatomy, such as: a set of points in a cut-file to autonomously modify the volume of bone to be removed; a set of virtual boundaries defined to haptically constrain a tool within the defined boundaries to modify the bone; a set of planes or drill holes to drill pins in the bone; or a graphically navigated set of instructions for modifying the tissue. The data generated from the planning computer 614 may be transferred to the device computer 616 and/or tracking computer 619 through a wired or wireless connection in the operating room (OR); or transferred via a non-transient data storage medium (e.g., a compact disc (CD), a portable universal serial bus (USB) drive) if the planning computer 614 is located outside the OR.

The device computer 616 in some inventive embodiments may be housed in the moveable base 608 and contain hardware, software, data and utilities that are preferably dedicated to the operation of the surgical device 602. This may include surgical device control, robotic manipulator control, the processing of kinematic and inverse kinematic data, the execution of registration algorithms, the execution of calibration routines, the execution of surgical plan data, coordinate transformation processing for determine the transformation T between the surgical plan, the bone B, and the surgical system 600, providing workflow instructions to a user, utilizing position and orientation (POSE) data from the tracking system 606.

The optional tracking system 606 of the surgical system 600 includes two or more optical receivers 630 to detect the position of fiducial markers (e.g., retroreflective spheres, active light emitting diodes (LEDs)) uniquely arranged on rigid bodies or integrated with a device itself. The fiducial markers arranged on a rigid body are collectively referred to as a fiducial marker array 632, where each fiducial marker array 632 has a unique arrangement of fiducial markers, or a unique transmitting wavelength/frequency if the markers are active LEDs. An example of an optical tracking system is described in U.S. Pat. No. 6,061,644. The tracking system 606 may be built into a surgical light, located on a boom, a stand 640, or built into the walls or ceilings of the OR. The tracking system computer 619 may include tracking hardware, software, data and utilities to determine the POSE of objects (e.g., bones B, surgical device 602) in a local or global coordinate frame. The POSE of the objects is collectively referred to herein as POSE data, where this POSE data may be communicated to the device computer 616 through a wired or wireless connection. Alternatively, the device computer 616 may determine the POSE data using the position of the fiducial markers detected from the optical receivers 630 directly.

The POSE data is determined using the position data detected from the optical receivers 630 and operations/processes such as image processing, image filtering, triangulation algorithms, geometric relationship processing, registration algorithms, calibration algorithms, and coordinate transformation processing. For example, the POSE of a digitizer probe 638 with an attached probe fiducial marker array 632b may be calibrated such that the probe tip is continuously known as described in U.S. Pat. No. 7,043,961. The POSE of the tool tip or tool axis of the tool 615 may be known with respect to a device fiducial marker array 632a using a calibration method as described in U.S. patent application Ser. No. 15/548,138. The device fiducial marker 632a is depicted on the manipulator arm 610 but may also be positioned on the base 608 or the end-effector assembly 611. Registration algorithms (e.g., iterative closest point) may be executed to determine the transformations T between a bone B, a fiducial marker array 632c or 632d, a surgical plan, and the surgical robot 602 using the registration method described herein.

The POSE data may be used by the computing system 604 during the procedure to update the POSE and/or coordinate transformation T of the bone B, the surgical plan, and the surgical robot 602 as the manipulator arm 610 and/or bone B move during the procedure, such that the surgical robot 602 can accurately execute the surgical plan. It should be appreciated that in certain embodiments, other tracking systems may be incorporated with the surgical system 200 such as an electromagnetic field tracking system or a 6-degree of freedom (DOF) mechanical tracking system. An example of a 6-DOF mechanical tracking system is described in U.S. Pat. No. 6,322,567. In a particular inventive embodiment, the surgical system 600 does not include a tracking system 606, but instead employs a bone fixation and monitoring system that fixes the bone directly to the surgical robot 602 and monitors bone movement as described in U.S. Pat. No. 5,086,401.

In specific embodiments, the aforementioned digitizer for digitizing the registration recovery markers (200, 300, 400) may be the mechanical digitizer 618 having a plurality of links and joints that track the position of a digitizer probe assembled thereto. In other embodiments, the aforementioned digitizer for digitizing the registration recovery markers (200, 300, 400) is a tracked digitizer probe 638 tracked by the tracking system 606.

### Other Embodiments

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. A method for registering a bone comprising:
installing two or more registration markers on an exposed portion of the bone in a first stage of a two-stage revision procedure;
generating an image data set of the bone to create imaged registration markers;
generating a surgical plan for implanting one or more secondary implants in a second stage of the two-stage procedure based on the image data;
registering the bone to the surgical plan and a computer-assist device using the two or more registration markers by:
digitizing a set of positions of the two or more registration markers to create digitized registration markers; and
mapping the set of positions of the digitized registration markers with a corresponding set of positions of the imaged registration markers.

2. A method for a two-stage revision procedure as to a failed primary implant in the bone of a patient, comprising:
exposing the bone through a surgical incision to gain access to the failed primary implant in a first-stage of the two-stage revision procedure;
removing the primary implant from the bone;
installing two or more registration markers on an exposed portion of the bone;
then suturing the surgical incision to end the first stage of the two-stage revision procedure;
generating an image data set of the bone to create imaged registration markers;
generating a surgical plan for implanting one or more secondary implants in a second stage of the two-stage procedure based on the image data set;
re-exposing the bone to begin a second stage of the two-stage revision procedure;
registering the bone to the surgical plan and a computer-assist device using the two or more registration markers by:
digitizing a set of positions of the two or more registration markers to create digitized registration markers; and
mapping the set of positions of the digitized registration markers with a corresponding set of positions of the imaged registration markers; and
preparing the bone of the patient according to the surgical plan with the aid of the computer-assist device to implant the one or more secondary implants in the bone of the two-stage revision procedure.

3. The method of claim 1 wherein the digitizing is accomplished with a digitizer probe having a probe tip.

4. The method of claim 1 further comprising removing the one or more registration markers from the bone and then suturing the re-exposed bone.

5. The method of claim 1 wherein the digitizing is accomplished with a measurement system of at least one of: an electro-mechanical sensor arm having a digitizer prove, an optical tracking system with an optically tracked probe, an electro-magnetic tracking system, an ultrasound tracking system, or an imaging system, said imaging system optionally being at least one of computed tomography (CT), X-ray, fluoroscopy, ultrasound, or magnetic resonance imaging (MRI).

6. The method of claim 2 wherein the surgical procedure is computer-assisted total joint replacement (TJR) surgery.

7. The method of claim 1 wherein the set of positions are digitized as one or more physical coordinates in three-dimensional space.

8. The method of claim 1 wherein at least one of said two or more registration markers further comprises a divot.

9. The method of claim 1 wherein at least one of the two or more registration markers is a groove recovery marker, the groove recovery marker further comprising a bone engaging portion, a proximal portion for engaging a driver tool, and a groove between the bone engaging portion and the proximal portion, and optionally wherein at least one of the two or more registration markers is asymmetric or has only a finite number of planes of symmetry.

10. The method of claim 1 wherein the digitizing is conducted with an electro-mechanical sensor arm having a digitizer probe that is integrated to a base of a robotic arm for a robotic surgical system to perform the surgical procedure on the bone.

11. The method of claim 1 further comprising generating a three-dimensional (3D) model of the bone for use in the surgical plan, optionally wherein the surgical plan comprises a desired positions and orientations (POSE) for the one or more secondary implants as planned by a surgeon in a pre-operative planning software program having the 3D model of the bone and computer-aided design (CAD) files of the one or more secondary implants stored therein.

12. The method of claim 1 wherein during the second stage of the two-stage revision procedure, the surgical plan is registered by determining a transformation T between the image data set and the bone via the one or more registration markers, optionally. wherein the transformation T is determined by mapping the position of the digitized registration markers present on the bone with the imaged registration markers in the image data set.

13. The method of claim 15 further comprising a set of point/vectors collected on the one or more registration markers are mapped to corresponding points/vectors of the imaged registration markers.

14. The method of claim 1 wherein the one or more registration markers act as registration recovery markers during bone preparation to re-register the bone in the event of bone motion.

15. The method of claim 1 wherein the one or more registration markers have an antibiotic or bone growth property on the bone, optionally wherein the property is provided by a coating on the surface of the one or more registration markers.

16. The method of claim 1 wherein the one or more registration markers are porous having a matrix infused with a substance diffusing therefrom and into the bone.

17. The method of claim 1 wherein the one or more registration markers are made of a biocompatible material that degrades overtime.

18. A system for performing the method of claim 1.

19. A system for performing the method of claim 2.
